(19) United States
(12) Patent Application Publication
(10) Pub. No.: US 2022/0098546 A1
(43) Pub. Date: Mar. 31, 2022

(54) MICROBIOTA-DERIVED POSTBIOTICS: ALTERNATIVE SUPPLEMENT TO FETAL BOVINE SERUM FOR CULTURED MEAT

(71) Applicant: Biftek Inc., Newark, DE (US)

(72) Inventors: Kamil Can Akcali, Ankara (TR); Erdem Erikci, Ankara (TR); Fadime Kiran, Ankara (TR)

(73) Assignee: Biftek Inc., Newark, DE (US)

(21) Appl. No.: 17/486,944

(22) Filed: Sep. 28, 2021

Related U.S. Application Data
(60) Provisional application No. 63/084,029, filed on Sep. 28, 2020.

(51) Int. Cl. C12N 5/00; C12N 1/20; C12N 1/12

(52) U.S. Cl. CPC C12N 5/0018; C12N 1/20; C12N 2500/30; C12N 2500/34; C12N 2500/84; C12N 1/12

(57) ABSTRACT

Fetal bovine serum (FBS) is considered a major supplement in culturing cells. Due to ethical issues, high costs, and batch-to-batch variations, designing an alternative to the FBS in cell culturing is important. A biological supplement including of microbiota-derived postbiotics (MD-PBs) obtained from a fermentation medium of beneficial microorganisms isolated from natural microbiota sources was formulated, processed, combined, and used as an alternative to the FBS in stimulating cell proliferation and growth. According to results obtained from different cell lines and primary cells, the MD-PBs and exopolysaccharides (EPSs) alone and/or Bovine Serum Albumin (BSA) and Sericin stimulated cell growth better than the FBS and, thus the MD-PBs and the EPSs alone and/or the BSA and Sericin are used as a possible alternative to the FBS in cell culture experiments and cultured meat technology.

### CROSS REFERENCE TO THE RELATED APPLICATIONS

This application is based upon and claims priority to U.S. Provisional Application No. 63/084,029 filed on Sep. 28, 2020, the entire contents of which are incorporated herein by reference.

### TECHNICAL FIELD

The present disclosure relates to a cost-effective and ready to use biological supplement comprising of microbiota-derived postbiotics (MD-PBs) and their combinations with Bovine Serum Albumin (BSA) and sericin, as an alternative source for Fetal Bovine Serum (FBS) in the production of cultured meat and other cell culture experiments.

### BACKGROUND

Cell culture technique has numerous applications in medicine, pharmacology, veterinary science, agriculture, etc. Among those applications, cultivated meat technology will be an alternative way of meat production that involves stem cell culturing and tissue engineering methods.

Meat cultivation by tissue engineering requires isolation and expansion of pluripotent, multipotent, or unipotent stem cells. Following the expansion, the stem cells are differentiated into various cells such as muscle cells. The differentiated cells to the muscle tissue are harvested for further processing as food.

Cells require a growth medium supplement to stay viable and proliferate. In a regular cell culture setup, FBS, also called Fetal Calf Serum (FCS), is used as a supplement, which is a rich source required for proliferation and growth. The FBS was introduced to be used in the cell culture to stimulate cellular proliferation and survival in the late 1950s. It was realized that the FBS contains essential materials for cell survival and growth such as hormones, vitamins, transport proteins, trace elements, and growth factors. Since its introduction, the FBS has been used as a universal supplement in the culture media for the human, animal, and insect cell lines in the research of pharmaceutic and biotechnology. However, the method of its collection sets another example of animal abuse since both cow and embryo are killed during this process. More than 800,000 liters of the FBS are produced every year worldwide, which corresponds to 2 million fetal bovines. In addition to ethical concerns, FBS is very expensive and the demand for the FBS is continuously increasing. Therefore, it is not practically preferable to use the FBS as the source of the growth factors in the cultivated meat production because of the cost (1, 3). Thus, it is important to reduce or replace the use of FBS with a cost-effective solution in cell culture experiments and cultivated meat production.

Microbiota is the assemblage of the living microorganisms present in a defined environment, and is considered as a growing research field. The complex and dynamic mutualistic relationship between the members of the microbiota influences a multitude of physiological functions in the host. In addition, a wide range of metabolites that are produced by microbiota members can modulate different cell signaling mechanisms. Nowadays, a new term called “postbiotics” (PBs) can be regarded as an umbrella term for all microbial fermentation components including short-chain fatty acids (SCFAs), functional proteins, extracellular polysaccharides (EPS), etc. In one aspect, the invention that is the subject of this closure provides novel compositions and formulations including PBs derived from the liquid fermentation medium of different beneficial microorganisms, which are isolated from different microbiota sources.

In another aspect, the invention provides novel formulations including EPSs semi-purified from the liquid fermentation medium of different beneficial microorganisms, with the combination of Bovine Serum Albumin (BSA) and Sericin or, alone. EPSs as PB compounds are known as extracellular biopolymers synthesized or secreted by microorganisms during their growth, and possess several bio-functional attributes like anti-oxidative, cholesterol-lowering, immunomodulatory, anti-aging, gut microbiota modulatory, anti-toxic, anti-biofilm effect, and antitumor effects at preclinical trials.

Bovine Serum Albumin (BSA) has been used to increase the growth stimulatory effect of FBS. BSA is the most abundant protein (comprises 55-62% of blood protein) in blood serum. Its physiological role is to regulate the colloidal osmotic pressure of blood, carrying the endogenous, as well as exogeneous molecules such as hormones, fatty acids, metabolites and drugs.

Sericin is a globular protein with the molecular weight of 200 kDa, consists of random coil and β-sheets. Amino acids that give the protein its hydrophilic character can lead to crosslinks and copolymerization with other polymers. It is obtained from the silkworm, Bombyx mori, which produces high amount of sericin and fibroin at the end of fifth larval instar to build a cocoon that provides the optimal conditions for larval metamorphosis. Sericin and fibroin makes up the 98% of the cocoon structure. It is an adhesive protein that joins two fibroin filaments to generate the silk thread. In the presence of sericin, the silk fiber is harder, while the absence of it makes the thread more fragile. It is also a biologically inert material with many applications in various industries such as biomedicine, food and cosmetics. Sericin was shown to have potential use in culture media, cryopreservation, wound healing, antitumor effect, and tissue engineering, as well as a putative food supplement, and a vehicle for drug delivery. Therefore, any innovation that can increase the efficiency of recombinant proteins, which in turn would decrease the required concentration of them in the growth medium will be considered as significant contribution for the price reduction of cultivated meat production and use of postbiotics may also contribute to this field.

### SUMMARY

Cell culture medium requires FBS as a supplement for culturing the cells. The present disclosure proposes the use of MD-PBs and their combinations with BSA and sericin, as an alternative to FBS in cell culture experiments and cultivated meat production.

The present disclosure provides a cost-effective and ready to use biological supplement, comprising microbial fermentation components of microbiota-originated microorganisms, abbreviated as MD-PB, wherein the sterile biological supplement is configured to be added to a culture medium with an aim of stimulation of cell proliferation and growth. Further, the present disclosure provides an animal free approach instead of FBS during cultivated meat production. This approach involves the isolation and identification of the beneficial microorganisms from different microbiota sources, incubating the microorganisms to obtain the fermentation components as MD-PB by centrifugation and filtration, lyophilizing the MD-PB, and adding the lyophilized MD-PB or their combinations to a culture medium for meat cultivation to stimulate the cell proliferation and growth. In addition, to augment the effect of PB solutions, we combined with BSA and sericin in the formulation of PBs.

Beneficial microorganisms present in the natural microbiota, such as lactic acid bacteria include species of the genera Enterococcus sp., Lactobacillus sp., Lactococcus sp., Leuconostoc sp., Oenococcus sp., Pediococcus sp., Streptococcus sp. and Weissella sp., Bifidobacteria (breve, lactis, and infantis), spore-forming bacilli and yeasts, have generally recognized as safe (GRAS) status from Food and Drug Administration (FDA). PB refers to metabiotic or biogenic products, or soluble factors such as metabolic byproducts secreted by live microorganisms such as SCFAs, peptides, teichoic acids, EPSs, organic acids, etc. PBs are good in terms of their safety dose parameters, long shelf life, resistance to hydrolysis by mammalian enzymes, and the content of various signaling molecules which may affect the diverse physiological processes.

The present disclosure provides an effective alternative source for FBS to culture primary cells and cell lines, which can solve the problems such as sustainability, animal welfare, and supply challenges. Any animal product used in the cell culture setup, such as the FBS, can be replaced by the MD-PBs and their combinations with BSA and sericin, as a novel biological supplement of the present disclosure which is an animal-free and food-grade formulation.

### BRIEF DESCRIPTION OF THE DRAWINGS

To more clearly illustrate the embodiments of the present disclosure, a brief description of the drawings is given below. The following drawings are only illustrative of some of the embodiments of the present disclosure and for a person of ordinary skill in the art, other drawings or embodiments may be obtained from these drawings without an inventive effort.

FIG. 1: Brief description of the protocol to obtain the MD-PBs in the present disclosure.

FIG. 2: Brief description of the primary skeletal muscle satellite cell isolation protocol to be used in the present disclosure.

FIG. 3A: Cell viability assessed with MTT assay in Caco2 upon supplement of different MD-PBs (50 μg/ml for Caco2 cells and 100 μg/ml for HEP40 cells) or 10% FBS after 72-hour of incubation [2A: Pediococcus acidilactici isolated from honey-bee gut microbiota; 29.1: Weissella cibaria isolated from honey-bee pollen microbiota; 29.3: Pediococcus pentosaceus isolated from honey-bee pollen microbiota; RI: Lactobacillus reuteri isolated from Spalax sp.-rodent gut microbiota; K5: Lactococcus lactis isolated from milk microbiota; M3.1: Lactobacillus kunkeei isolated from honey-bee gut microbiota; F10.1: Lactobacillus plantarum isolated from human gut microbiota], ns: non-significant; *p<0.05; **p<0.005; ***p: 0.0005.

FIG. 3B: Cell viability assessed with MTT assay in HEP40 upon supplement of different MD-PBs (50 μg/ml for Caco2 cells and 100 μg/ml for HEP40 cells) or 10% FBS after 72-hour of incubation [2A: Pediococcus acidilactici isolated from honey-bee gut microbiota; 29.1: Weissella cibaria isolated from honey-bee pollen microbiota; 29.3: Pediococcus pentosaceus isolated from honey-bee pollen microbiota; RI: Lactobacillus reuteri isolated from Spalax sp.-rodent gut microbiota; K5: Lactococcus lactis isolated from milk microbiota; M3.1: Lactobacillus kunkeei isolated from honey-bee gut microbiota; F10.1: Lactobacillus plantarum isolated from human gut microbiota], ns: non-significant; *p<0.05; **p<0.005; ***p<0.0005.

FIG. 4: Cell viability was assessed with MTT assay in LX2 cells upon supplement of different exopolysaccharides (EPS) in either 100 μg/ml or 10 μg/ml or 11 μg/ml concentrations or 10% FBS after 72-hour of incubation [15.1: Weissella confusa isolated from honey-bee pollen microbiota; 18.3: Weissella cibaria isolated from honey-bee pollen microbiota], ns: non-significant; *p<0.05; **p<0.005; ***p<0.0005.

FIG. 5: Cell viability was assessed with MTT assay in primary bovine satellite cells (BSC) at passage 4 upon supplement of EPS-2A in different combinations with BSA (400 μg/ml), and sericin (500 μg/ml) or 10% FBS after 72-hour of incubation, *p<0.05; p<0.005; ***p<0.0005: ****p<0.0001.

FIG. 6A: Characterization of BSCs by immunofluorescence staining (100× magnification), Pax3/7.

FIG. 6B: Characterization of BSCs by immunofluorescence staining (100× magnification), MyoD b).

FIG. 7A: Morphological observation of BSCs (10× magnification). Morphology of BSCs supplemented with 10/% FBS.

FIG. 7B: Morphological observation of BSCs (10× magnification). Morphology of BSCs supplemented with 12.5 μg/mL 2A-EPS.

### DETAILED DESCRIPTION OF THE EMBODIMENTS

The technical solutions of the present disclosure will be clearly and completely described below. The embodiments described are only some of the embodiments of the present disclosure, rather than all of the embodiments. All other embodiments that are obtained by a person of ordinary skill in the art based on the embodiments of the present disclosure without an inventive effort shall be covered by the protective scope of the present disclosure.

To our knowledge, there is no study of MD-PBs applied in the cultivated meat production. Unless specifically defined, all technical and scientific terms used herein have the same meaning as commonly understood by a skilled artisan in enzymology, biochemistry, cellular biology, molecular biology, and the medical sciences.

The present invention, which discloses a sterile biological supplement abbreviated as MD-PB, is described in detail below.

The present disclosure comprises, alone or in the combined form of MD-PBs obtained from a strain from the genus of Enterococcus sp., Lactobacillus sp., Lactococcus sp., Leuconostoc sp., Oenococcus sp., Pediococcus sp., Streptococcus sp. and Weissella sp., Bifidobacteria (breve, lactis, and infantis), spore-forming bacilli and yeasts which are all accepted as environmentally friendly components for animal and human health and approved by the FDA as GRAS.MD-PBs obtained from different strains of beneficial bacteria and yeasts were added to the culture mediums in different concentrations and different formulations with BSA and Sericin or alone, then screened for their possible effects on the stimulation of the growth of primary cells and cell lines using cell cytotoxicity assay (MTT assay), cellular morphological analysis and immunofluorescence methods. In addition to MD-PBs, EPSs in PBs were also tested. Our results showed that, promising MD-PBs and EPSs alone and/or BSA and sericin stimulated cell growth better to that of FBS and, thus they can be used as a possible alternative to FBS in cell culture experiments and cultured meat technology.

The MD-PB supplement can be in a lyophilized powder form, and be soluble in a sterile culture medium or water.

The embodiments of the present disclosure are described in detail below.

### 1. Isolation and Identification of the Beneficial Microorganisms

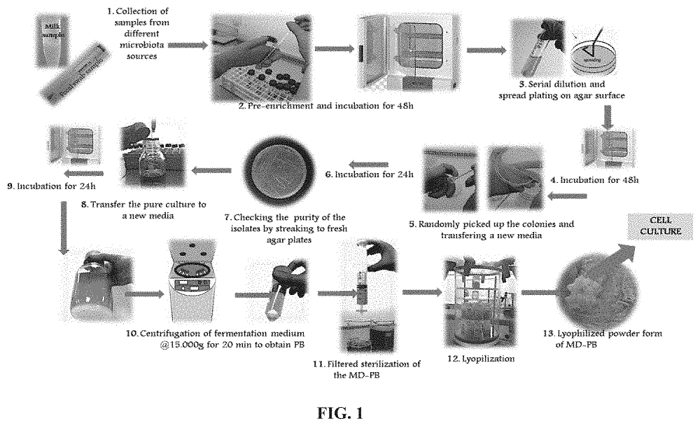

Different samples taken from different microbiota sources were pre-enriched in the media such as MRS (De Man, Rogosa and Sharpe agar), M-17, TOS-MUP (TOS Propionate Agar & Lithium Mupirocin), Bacillus selective media, and yeast growth media (FIG. 1; Steps 1-2), and various lactic acid bacteria, bifidobacteria, spore-forming bacilli, and yeasts were isolated using microbiological methods (FIG. 1; Steps 3-7). Briefly, one milliliter of pre-enriched cultures was mixed with 9 mL sterile physiological saline (0.85% w/v, NaCl) to make an initial dilution. Serial dilutions were carried out for each sample and then 1 ml of the appropriate dilution was spread plated on agar plates (FIG. 1; Step 3). Following the incubation at 30-37° C. for 48 h (FIG. 1; Step 4) under aerobic or facultative anaerobic conditions in a microbiological jar, colonies with distinct morphological differences (based on color, shape, or surface type) were randomly selected, picked up, and then purified using streak plating on agar surface (FIG. 1; Steps 5-7). Finally, Gram-positive, catalase-negative, and non-motile microorganisms and yeasts were maintained in 10% (w/v) skim milk with 50% glycerol and stored at −80° C., for their long-term storage. Selected isolates were then identified using 16S-18S gene sequencing.

### 2. Collection, Formulation, and Processing of MD-PMs

The resulting isolates were grown in the selective media in aerobic conditions at 30-37° C. for 24 hours to reach 10⁸ cfu/ml (colony-forming units per milliliter)(FIG. 1; Steps 8-9). Following the incubation, the fermentation medium containing PB was obtained from the overnight culture by centrifugation at 15.000 g for 20 minutes and passed through the sterile membrane filters with 0.22 μm pore diameter (FIG. 1; Steps 10-11). Finally, the MD-PBs were lyophilized (0.120 mB vacuum pressure and −58° C. converter temperature)(FIG. 1; Steps 12-13). The powder MD-PBs were then diluted in different doses with the range of 0.01% to 10%, 0.1% to 10%, 1.0/0 to 10%, or 1.0% to 5.0% by weight to be used in the cell culture experiments.

In addition to MD-PBs, EPSs in PBs were purified from the microorganisms to be used as an alternative supplement instead of FBS, in cell culture experiments. To obtain EPS; the resulting isolates were grown in the selective media in aerobic conditions at 30-37° C. for 24 hours to reach 10⁸ cfu/ml. Following the incubation period, the fermentation medium was boiled for 15 minutes and the MD-PBs obtained after centrifugation, were used as an EPS source. MD-PBs treated with 20% (v/v) trichloroacetic acid were incubated at 4° C. for 2 hours under shaking conditions, and then the pellet was separated via centrifugation (8.000 g, 30 min, 4° C.). The supernatant was mixed with two volumes of 95% cold ethanol, then kept at 4° C., overnight. Finally, EPS was collected following the centrifugation at 10.000 g for 30 min at 4° C., and then lyophilized to obtain dried EPS. For the combinatorial studies, recombinant BSA and sericin were obtained commercially (Sigma-Aldrich).

### 3. Cell Culture

The proliferative effects of the different concentrations of the lyophilized MD-PBs derived from the different microorganisms that originated from natural microbiota were tested and compared to those of FBS. Cell lines such as LX2, HEP40, Caco2, and C2Cl2 were maintained in T75 culture flasks containing Dulbecco's Modified Eagle Media (DMEM) supplemented with 1% penicillin-streptomycin. The cells were incubated at 37° C. with 5% CO₂. All cells were passaged at 75-80% confluence.

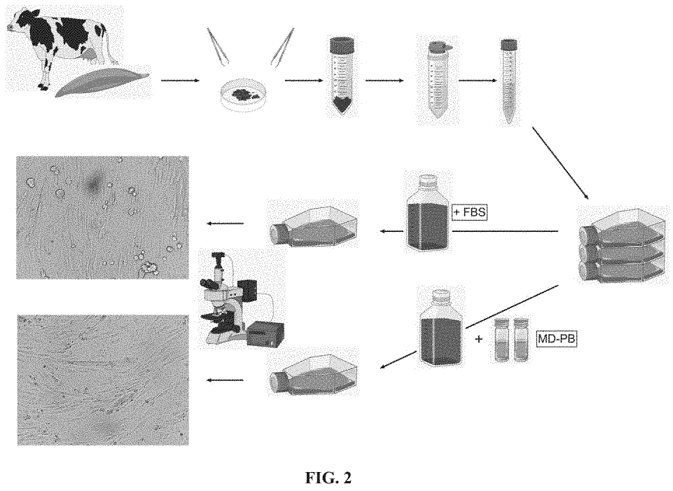

Primary skeletal muscle satellite cell isolation (FIG. 2): The skeletal muscle biopsy was gently cut into small pieces with the help of tweezers. The use of sharp tools was avoided. The shredded tissue was transferred to a 15 ml falcon containing 1 ml of digestion medium (Table 1). The lids of the falcons were wrapped with parafilm and placed in a 37° C. water bath. Every 10 minutes the falcons were shaken gently. The incubation period varies according to the age of the animal from which the sample was taken and the freshness of the tissue. For this reason, the turbidity of the tube should be carefully monitored. For example, 45 minutes of incubation was sufficient for the dissociation of the freshly dissected tissue from a 6-month-old bovine fetus. Upon the completion of the incubation, the falcons were vortexed for 5 seconds, and 1 ml of neutralizing medium (Table 1) was added into the tubes. The digested tissues were gently resuspend using a 1000 μl micropipette tip that was cut at the bottom. A successful digestion is denoted by the complete dissolution of the tissue. Next, a pre-moistened 40 μm tissue filter was placed on a 50 ml falcon and damped again. The tissue suspension was gently poured onto the filter. Since the 40 μm filters easily get clogged during the process, the suspension should be passed through the filter very slowly. The resulting cell suspension was centrifuged at 300×g for 5 minutes. The supernatant was discarded, and the pellet was dissolved in a 2 ml culturing medium (Table 1). Cells were counted and seeded onto Matrigel-coated plates accordingly. The medium was changed after 48 hours.

TABLE 1
Medium ingredients for the primary muscle satellite
cell isolation from bovine skeletal muscle biopsy.

| | |
|---|---|
| Digestion medium | DMEM (w/o FBS) |
| | 1% p/s |
| | 25 mM CaCl₂ |
| | Collagenase II (>108 U/ml) |
| Collection, washing, and | 1 × PBS |
| mincing solution | 1% p/s |
| Neutralizing and culturing | DMEM |
| medium | 1% p/s |
| | 20% FBS |

Matrigel coating: Stock Matrigel was dissolved in FBS-free medium to reach a concentration of 10%. The 10% matrigel solution was dispensed into microcentrifuge tubes as 1 ml aliquots and the tubes were immediately brought to −20° C. The final Matrigel coating concentration was 0.1%. The solution was made by diluting the 10% Matrigel stock in a serum-free medium. Enough Matrigel solution was poured into the culture dish or well, incubated for 30 seconds and it was transferred to the other well or container. Matrigel-coated plates were incubated for 20 minutes at room temperature in a sterile laminar cabinet with the lid open. At the end of 20 minutes, the lid was closed, and the cells were not taken into the incubator until seeding.

Cell viability assay: Cells were trypsinized, counted and then seeded into 96-well plates (1×10 W cells/well). The MD-PBs were applied in different concentrations as described in figure legends and incubated at 37° C., 5% CO₂ for 72 hours. MTT assay, which is a colorimetric assay for assessing cell metabolic activity, was performed to assess cell viability and proliferation. Tetrazolium salt (3-(4,5-dimethylthiazol-2-yl)-2,5-diphenyltetrazolium bromide salt) at concentration of 5 mg/ml was added into wells to achieve the final concentration of 0.5 mg/ml. Formazan (artificial chromogenic products of the reduction of tetrazolium salts by dehydrogenases and reductases) producing reaction was performed at 37° C. for at least 2 hours. Following the incubation, the medium was removed. Formazan crystals were dissolved in 100 μl of dimethyl sulfoxide (DMSO). The absorbance of the formazan product was measured at 590 nm by a microplate reader.

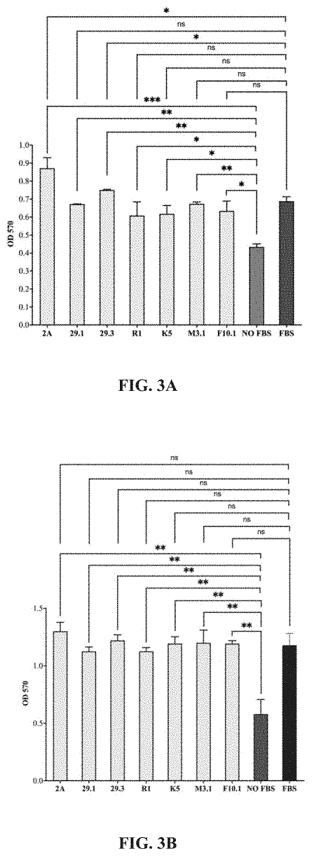

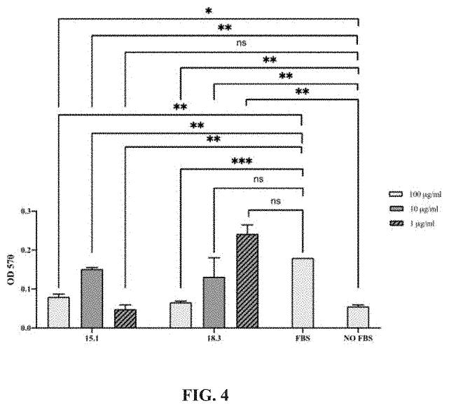

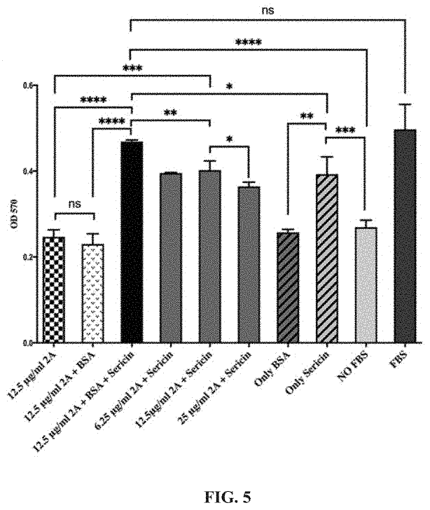

The results showed that the MD-PBs and EPSs of the present disclosure exhibited cell growth-promoting effects on the cell lines (FIGS. 3A, 3B, 4) and primary cells (FIG. 5). All the PBs tested showed a better proliferation capacity compared to No FBS group (FIGS. 3A, 3B). In addition, all PBs have similar proliferative effect when compared to FBS group (FIGS. 3A, 3B). Amongs them, PBs derived from 2A (Pediococcus acidilactici isolated from honey-bee gut microbiota) and 29.3 (Pediococcus pentosaceus isolated from honey-bee pollen microbiota), showed the most prominent proliferative effect in Caco2 cells (FIG. 3A). We also tested the effect of EPS on the LX2 cell line (FIG. 4). Our results showed that EPS extracted from 18.3 (Weissella cibaria isolated from honey-bee pollen microbiota) has similar proliferative effect compared to FBS group. Moreover, this effect was concentration-dependent, and was significantly different to that of No FBS group. In addition, EPS extracted from 2A and its combinations with BSA and sericin, have shown statistically significant (p. 0.0001) growth rate compared to No FBS, and no difference compared to FBS (FIG. 5).

### 4. Morphological Observation and Immunostaining of Primary Bovine Muscle Satellite Cells (BSCs)

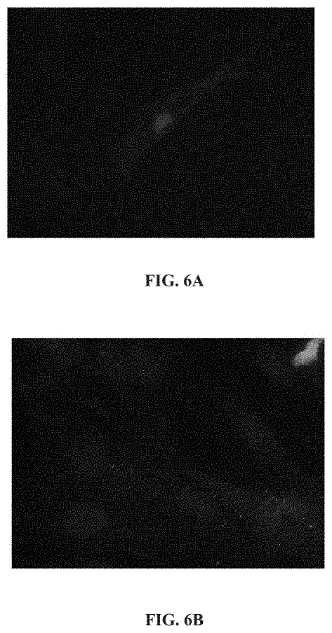

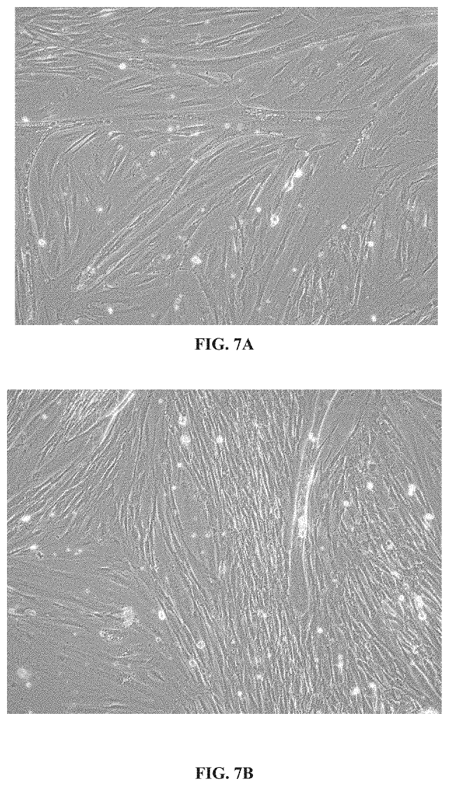

Characterization was carried out by immunofluorescence staining with Pax 3/7 and MyoD antibodies (FIGS. 6A, 6B). The morphological characteristics of the cells were observed under a tissue culture microscope PrimoVert (Zeiss). EPS-treated BSCs showed no morphological change in comparison with the untreated group (FIGS. 7A, 7B).

Initial FBS supplement is necessary for cell attachment in cell culture flasks. Therefore, we treated the cells with either EPS or MD-PBs after cells were attached overnight with FBS-supplemented medium. We observed that cells were not be able to attach without the FBS supplement even if they were supplemented with our MD-PBs.

The present disclosure provides an alternative supplement to add to the culture instead of FBS. The proliferative effect of the MD-PBs or EPSs can be associated with the inactivation of pro-apoptotic signals and the activation of anti-apoptotic signals.

Further, the present disclosure provides the “natural” and “low cost” alternative to FBS as a single product, and also has a proliferative and growth promoting effect on the primary cells and cell lines. MD-PBs or EPSs of the present disclosure were produced in the form of the lyophilized powder formulation which easily dissolves in water or ideal mediums, and can be directly incorporated into cell culture applications. Both MD-PBs or EPSs also preserve their functional properties in production, transportation, storage, and application processes.

### 5. Statistical Analysis

All assays were performed with three independent experiments and each measurement was carried out in triplicate. Data were analyzed by student's unpaired t-test. p<0.05 was used to indicate a significant difference.

What is claimed is:

1. A biological supplement, comprising:
postbiotics of beneficial aerobic and facultative aerobic/anaerobic microorganisms isolated from natural microbiota,
wherein the biological supplement is configured to be added to a culture medium with an aim of a stimulation of cell growth and proliferation.

2. The biological supplement according to claim 1, comprising postbiotics derived from a strain of lactic acid bacteria.

3. The biological supplement according to claim 1, comprising postbiotics derived from a strain of Bifidobacteria.

4. The biological supplement according to claim 1, comprising postbiotics derived from a strain of spore-forming bacilli.

5. The biological supplement according to claim 1, comprising postbiotics derived from a strain of yeasts.

6. A method of collection, formulation, combination and processing of the postbiotics, comprising the biological supplement of claim 1.

7. The method according to claim 6, comprising the postbiotics derived from a strain of lactic acid bacteria.

8. The method according to claim 6, comprising the postbiotics derived from a strain of Bifidobacteria.

9. The method according to claim 6, comprising the postbiotics derived from a strain of spore-forming bacilli.

10. The method according to claim 6, comprising the postbiotics derived from a strain of yeasts.

11. The biological supplement according to claim 1, comprising exopolysaccharides wherein the postbiotics, derived from a strain of lactic acid bacteria.

12. The biological supplement according to claim 1, comprising exopolysaccharides, wherein the postbiotics, derived from a strain of Bifidobacteria.

13. The biological supplement according to claim 1, comprising exopolysaccharides, wherein the postbiotics, derived from a strain of spore-forming bacilli.

14. The biological supplement according to claim 1, comprising exopolysaccharides, wherein the postbiotics, derived from a strain of yeasts.

15. The biological supplement according to claim 1, comprising combination of Bovine Serum Albumin.

16. The biological supplement according to claim 1, comprising combination of Sericin.

17. The biological supplement according to claim 1, comprising an alternative supplement for Fetal Bovine Serum to be used in cultivated meat production, wherein the cultivated meat production is a primary muscle cell culturing.

18. The biological supplement according to claim 1, comprising an alternative supplement for Fetal Bovine Serum in transformed cell lines culture medium.

19. The biological supplement according to claim 1, wherein the biological supplement is in a lyophilized powder form, and soluble in a sterile medium or water.
